(19) United States
(12) Patent Application Publication
(10) Pub. No.: US 2025/0022550 A1
(43) Pub. Date: Jan. 16, 2025

(54) SYSTEMS AND METHODS FOR PREDICTING PET AMYLOID BIOMARKER STATUS USING MULTIMODAL DIGITAL COGNITIVE ASSESSMENTS

(71) Applicants: Karl Thompson, Cambridge, MA (US); Connor Higgins, Boston, MA (US); Russell Banks, Boston, MA (US); Ali Jannati, Boston, MA (US); Joyce Gomes-Osman, Boston, MA (US); John Showaiter, Boston, MA (US); Sean Tobyne, Boston, MA (US); Alvaro Pascual-Leone, Boston, MA (US); David Bates, Boston, MA (US)

(72) Inventors: Karl Thompson, Cambridge, MA (US); Connor Higgins, Boston, MA (US); Russell Banks, Boston, MA (US); Ali Jannati, Boston, MA (US); Joyce Gomes-Osman, Boston, MA (US); John Showaiter, Boston, MA (US); Sean Tobyne, Boston, MA (US); Alvaro Pascual-Leone, Boston, MA (US); David Bates, Boston, MA (US)

(21) Appl. No.: 18/768,458

(22) Filed: Jul. 10, 2024

Related U.S. Application Data
(60) Provisional application No. 63/526,085, filed on Jul. 11, 2023.

(51) Int. Cl. G16H 10/20; A61B 6/50; G06N 5/022

(52) U.S. Cl. CPC G16H 10/20; G06N 5/022; A61B 6/501

(57) ABSTRACT

Systems and methods are disclosed for predicting a biomarker status. A method of predicting a biomarker status comprises administering a battery of assessments to a patient; collecting multimodal data based on one or more responses to the battery of assessments from the patient; extracting one or more feature sets from the one or more response; providing the one or more feature sets to a trained machine learning model; predicting, using the trained machine learning model, a status of a biomarker of the patient; providing the prediction into a recommendation engine; determining one or more interventions based on the prediction, wherein the one or more interventions include values to the patient; and providing the one or more interventions as output.

### RELATED APPLICATION(S)

This application claims the benefit of priority to U.S. Provisional Application No. 63/526,085, filed Jul. 11, 2023, which is incorporated herein by reference in its entirety.

### TECHNICAL FIELD

The invention relates generally to predicting beta amyloid status, and, in particular, to systems and methods for predicting beta amyloid status using a multimodal digital cognitive assessment.

### BACKGROUND

The most common and well-established methods to detect beta-amyloid status (αβ±), a biomarker associated with Alzheimer's disease (AD), in the brain are extracting the cerebrospinal fluid through a spinal tap or using a positron emission tomography (PET) brain image. While accurate and relatively safe for patients, these methods are prohibitively expensive, require substantial amounts of time and resources to perform, not universally available, and, in the case of the spinal tap, physically invasive and potentially painful. Currently, innovation in this space seeks to identify αβ± by analyzing blood samples using recently developed specialized laboratory assessments. Importantly, the current solutions require specialized medical facilities, hardware, and often immobile and inconvenient requiring specific instruments to conduct. However, the availability of this method and its accuracy in diverse populations is not widely available and likely a limiting factor to its use.

The present disclosure describes a proposed system and methods for predicting an individual's PET αβ± status using multimodal digital cognitive assessment technology that provides a quick, inexpensive, digital, noninvasive solution for predicting αβ± obtained from the multimodal digital assessment technology. This multimodal prediction method includes the analysis of the DCTclock™, an FDA-registered Class II medical device, a delayed 3-word verbal memory test, and analysis of temporal speech and acoustic voice features and self-reported survey questions that capture key lifestyle and health factors for cognitive impairment/dementia. The temporal and acoustic speech and voice analysis are conducted on recorded audio, rendering this AD biomarker prediction model a multimodal analysis of graphomotor behavior (DCTclock), delayed verbal recall, and voice/speech features (spoken recall audio analysis).

### SUMMARY

According to certain aspects of the present disclosure, systems and methods are disclosed for predicting beta amyloid status using a multimodal digital cognitive assessment.

In one embodiment, a method for predicting a biomarker status comprises administering a battery of assessments to a patient, collecting multimodal data based on one or more responses to the battery of assessments from the patient, extracting one or more feature sets from the one or more response, providing the one or more feature sets to a trained machine learning model, predicting, using the trained machine learning model, a status of a biomarker of the patient, providing the prediction into a recommendation engine, determining one or more interventions based on the prediction, wherein the one or more interventions include values to the patient, and providing the one or more interventions as output.

In some embodiments, the one or more feature is extracted using a first order measure. In some embodiments, the first order measure comprises a feature extracted from the response and organized by a modality associated with the feature.

In some embodiments, the one or more feature is extracted using a second order measure. In some embodiments, the second order measure comprises extracting an embedded characteristic of the response and associating the characteristics with a modality.

In some embodiments, the method further comprises determining a health condition based on the status of the biomarker.

In some embodiments, determining the one or more interventions comprises thresholding the biomarker status.

In some embodiments, the one or more interventions include a holistic state of the prediction. In some embodiments, the holistic state of the prediction comprises receiving additional data associated with the patient, and weighing the prediction with the additional data.

In some embodiments, the trained machine learning model is trained with a subset of feature sets as an input. In some embodiments, the subset of feature sets includes a delayed recall score, a composite clock score, an average speed score, an oscillatory motion feature score, and a maximum speed score.

In some embodiments, the trained machine learning model is a regression model. In some embodiments, the regression model is a logistic regression classifier.

In some embodiments, the battery of assessments includes at least one of a digital clock and recall assessment and a DCTclock assessment.

In some embodiments, the collecting the multimodal data comprises collecting data from at least a touchscreen, a microphone, a webcam, and/or a stylus.

In some embodiments, the biomarker comprises a beta-amyloid.

In some embodiments, providing the one or more interventions as output comprises transmitting the one or more interventions to a computing device associated with a clinician.

In some embodiments, the one or more interventions comprise a score for a plurality of categories and a recommendation for each category.

In some embodiments, the recommendation includes a physical or mental evaluation of the patient.

In some embodiments, the method further comprises receiving patient demographic and medical history from the patient.

In some embodiments, the battery of assessments is conducted on a mobile computing device.

In another embodiment, a system for predicting a biomarker status comprises a computing node comprising a computer readable storage medium having program instructions embodied therewith, the program instructions executable by a processor of the computing node to cause the processor to perform a method comprising administering a battery of assessments to a patient; collecting multimodal data based on one or more responses to the battery of assessments from the patient; extracting one or more feature sets from the one or more response; providing the one or more feature sets to a trained machine learning model; predicting, using the trained machine learning model, a status of a biomarker of the patient; providing the prediction into a recommendation engine; determining one or more interventions based on the prediction, wherein the one or more interventions include values to the patient; and providing the one or more interventions as output.

In an alternate embodiment, a computer program product for predicting a biomarker status comprises a computer readable storage medium having program instructions embodied therewith, the program instructions executable by a processor to cause the processor to perform a method comprising administering a battery of assessments to a patient; collecting multimodal data based on one or more responses to the battery of assessments from the patient; extracting one or more feature sets from the one or more response; providing the one or more feature sets to a trained machine learning model; predicting, using the trained machine learning model, a status of a biomarker of the patient; providing the prediction into a recommendation engine; determining one or more interventions based on the prediction, wherein the one or more interventions include values to the patient; and providing the one or more interventions as output.

### BRIEF DESCRIPTION OF THE DRAWINGS

The accompanying drawings, which are incorporated into and constitute a part of this specification, illustrate various exemplary embodiments and together with the description, serve to explain the principles of the disclosed embodiments.

FIG. 1 is a block flow diagram illustrating an example technique for predicting a beta amyloid status of a patient, according to embodiments of the present disclosure.

FIG. 2 illustrates an example process for data used to train a machine learning model, according to embodiments of the present disclosure.

FIG. 3 is a further example workflow diagram predicting an Aβ± status, according to embodiments of the present disclosure.

FIG. 4 is a schematic of an example of a computing node, according to embodiments of the present disclosure.

### DETAILED DESCRIPTION

Reference will now be made in detail to the exemplary embodiments of the present disclosure, examples of which are illustrated in the accompanying drawings. Wherever possible, the same reference numbers will be used throughout the drawings to refer to the same or like parts.

The systems, devices, and methods disclosed herein are described in detail by way of examples and with reference to the figures. The examples discussed herein are examples only and are provided to assist in the explanation of the apparatuses, devices, systems, and methods described herein. None of the features or components shown in the drawings or discussed below should be taken as mandatory for any specific implementation of any of these devices, systems, or methods unless specifically designated as mandatory.

Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

As used herein, the term “exemplary” is used in the sense of “example,” rather than “ideal.” Moreover, the terms “a” and “an” herein do not denote a limitation of quantity. but rather denote the presence of one or more of the referenced items.

Current tests for Aβ± status require PET scans, spinal taps drawing cerebrospinal fluids, and other solutions such as drawing blood. Drawbacks of these tests include the substantial cost, required resources and skilled-personnel effort, lack of universal availability, long completion times, and considerable invasiveness. There is a need for an inexpensive, universally applicable, quick, and noninvasive solution. A mobile solution would open possibilities for more generalized and widely available screening.

Using AI enabled automatic analysis of data captured during performance of the DCTclock and delayed recall assessment components, embodiments of the present disclosure are able to predict differing levels of the presence of αβ± in cognitively unimpaired older adults. There are two important distinguishing factors of these algorithms contributing to their uniqueness. First is the specific collection of measures used in a multimodal approach and their specific application to the prediction of amyloid status. An individual's assessment performance is analyzed by stages of algorithms related to the specific assessments (e.g., DCTclock, DCR, and Trail Making Test A&B) and models to produce a set of features (see Table 1 below).

Compared to currently available solutions, the Aβ prediction as described by embodiments of the present disclosure is inexpensive, widely applicable to diverse populations, quick, and physically non-invasive. Different from most other single modality methods used to detect neural biomarkers of Alzheimer's Disease Related Dementia (ADRD), embodiments of the present disclosure consider multiple modalities to predict the presence of important ADRD biomarkers. This allows physicians to identify individuals who are most likely to have elevated Aβ burden in their brain. This prediction is valuable for identifying and guiding essential next steps in the diagnosis and treatment of patients with mild cognitive impairment and early stages of ADRD. Embodiments of the present disclosure can be used by a mobile device, and thus, provides the opportunity for screening a wider and more diverse population who may not have access to the level of healthcare facility required for existing solutions (e.g., PET facilities, specialized blood assay hardware, etc.).

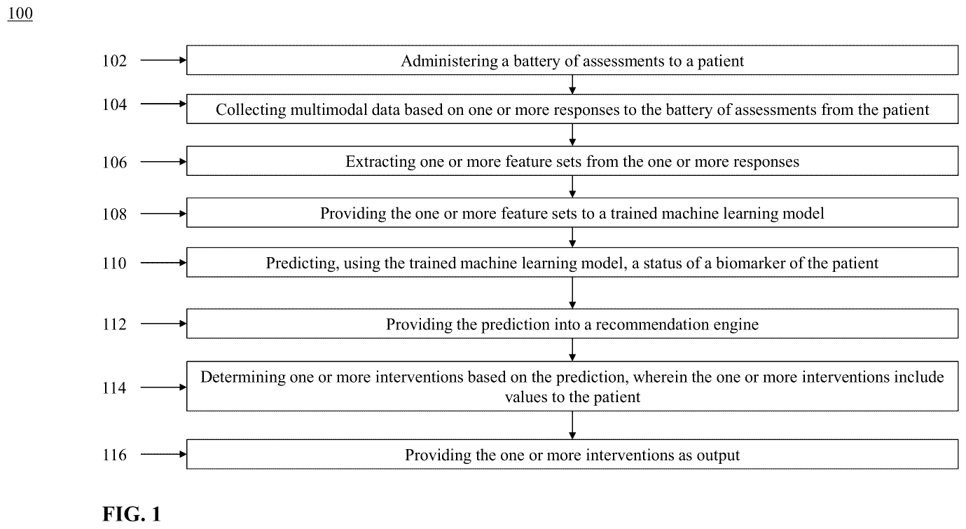

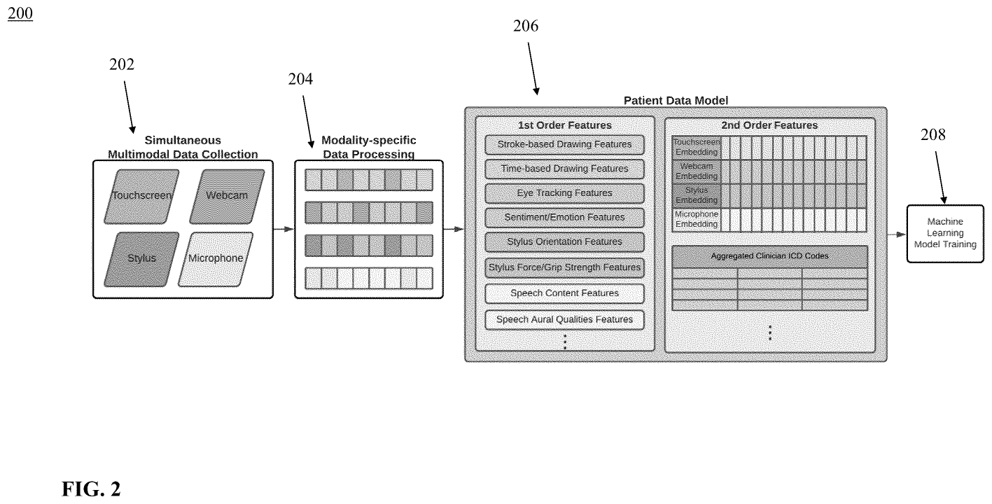

FIG. 1 is a block flow diagram illustrating an example technique 100 for predicting a beta amyloid status of a patient. The stages of analysis for technique 100 follow a general workflow process as follows:
A battery of assessments may be administered to a patient subject and multimodal data may be collected of their individual responses (steps 102 and 104);
Additional data from electronic health records, clinician feedback, and others may be incorporated and combined with the multimodal assessment data;
Feature sets (or “features”) from each patient may be extracted and/or derived using 1st linear and 2nd order non-linear measures (step 106);
The feature sets may be provided/input to a trained machine learning model, which can then predict biomarkers and/or other cognitive health conditions of the subject (steps 108 and 110);
The predicted biomarkers, cognitive health conditions, and potentially, the feature sets may be provided/input to a recommendation engine, as further shown and described with relation to FIG. 2 (step 112 in method 100). The recommendation engine may consider the holistic state of the prediction that it is provided and may recommend one or more interventions that it predicts will produce customized outcomes that positively affect the desired changes in those predictions and values that are unique to the individual patient (step 114). These unique predictions may include values related to memory, motor, cognition, hearing, sleep, and vision. This may also include predictions related to patient demographics, medical history, and survey responses entered into the patient platform The recommendation engine may also output the recommended one or more interventions to the clinician (step 116);
The clinician can perform the intervention recommended by the engine;
After a period of time, the assessments may be readministered to determine if the intervention had the desired outcome effects on the patient(s).

The features extracted may be used as predictors of Aβ in a pre-trained machine learning algorithm, similar to what is described in FIG. 2, to produce a thresholded likelihood estimate (i.e., a positive or negative ratio of sensitivity/specificity) or point estimate (e.g., a centiloid value: 0-100 scale of presence/absence of the target biomarker) of Aβ biomarker (PiB; described below) presence.

In an initial stage of the analysis described above, a patient may be directed to complete a battery of assessments, including a Digital Clock and Recall (DCR) assessment. among others. For example, a DCR assessment may include voice and speech analysis score is added to a scoring algorithm where the speech timing and acoustic voice is added as an addition to a 3-word recall task. The DCR assessment involves a patient drawing two clocks on a tablet. In particular, in the DCR assessment, a patient is asked to “freeform” draw a clock and then asked to draw a clock with a particular time using the traditional two hands of the clock followed by a verbal recall test. The algorithm tracks the patient's progress at 120x/second and correlates the tracked data to other existing data related to normal cognitive function and potential cognitive impairment. As another example, a Trails A&B assessment involves asking a patient to connect dots between letters (A-Z) and numbers (1-9 et. al.) that are interspersed and scattered throughout a display screen. The tracked patient responses and lines drawn may be correlated with data related to normal and abnormal cognitive function. As yet another example, a DCTclock analysis involves measuring how a pen or stylus stroke moves as compared to a smooth pen movement during the drawing process. The results of this assessment may be captured and/or included when the pen or stylus is used, regardless of which test/assessment is being completed. The battery of assessments can be conducted on a tablet, smartphone, or any other processing device where the patient is able to respond. Embodiments of the present disclosure use established and proven norms published in literature for iterations of voice and speech testing. It is contemplated that alternative embodiments can incorporate a machine learning process that does not rely on published norms, and instead will make use of both (1) standardized threshold based assessments; and (2) sample based thresholding for an individual patient determined by the machine learning model.

For the delayed three-word verbal memory test/assessment, patients are presented with three words to remember. Immediately following encoding of the three word recall task, patients complete the DCTclock drawing tasks, as described above. Following the completion of the DCTclock drawings (typically requiring 2-3 minutes to complete and thus, acting as the delay for the verbal recall task), patients are asked to recall the 3 words originally encoded in the immediate recall condition.

Verbal memory has been researched extensively as a means of differentiating and classifying disease populations from healthy individuals. Many of the most common word recall tasks used in clinical practice in the United States are derived from classic neurologic assessments. For example, the Mini-Cog™ is a brief pencil-and-paper screening tool commonly used to assess cognition in populations with known or suspected cognitive impairment. This tool pairs immediate and delayed word recall with a clock drawing task to assess memory, attention, and executive functioning. Traditionally, the Mini-Cog provides six, 3-word sets so that when the test is administered at different time points, different word sets can be used to avoid learning and carry-over effects. However, while appropriate for American English speakers, some of the words used in the recall task have less relevance in other languages or are overly complex, and are subsequently more difficult to remember. Accordingly, a new, cross-linguistically applicable set of words for use in word recall sets is included in the battery of assessments. In addition to being able to use the same sets of words across languages, the goal was to create a set of words that are commonly occurring (as determined by word frequency) and of similar speech sound (phoneme) complexity. To perform more sophisticated speech and voice production analyses, vowel diversity was a secondary focus of the new word set; recently, researchers have examined specific motor speech features extracted from acoustic signals of vowels in words to differentiate disease populations from healthy individuals. These analyses require a diverse set of vowel productions, including both central (produced with the tongue at rest in the center of the mouth) and non-central (requiring some movement of the tongue to produce). Thus, more can be inferred about speech motor ability, cognition, and vocal health and quality. Table 1 describes the important features of the original and new word sets in terms of the average normalized frequency of occurrence, phonemic complexity index, and counts of central and non-central vowels.

The battery of assessments, such as those described above, can include a range of tests designed to characterize the patient's cognitive ability and physiology. After the patient completes the DCR assessment within the battery, the resulting raw data may be analyzed and objective scoring metrics may be produced to describe various aspects of the feature sets gathered (see Table 1, below) of the patient's performance during the assessment. Embodiments of the present disclosure can incorporate five metrics/features that correlate with αβ± status: Delayed Recall score, DCTclock Spatial Reasoning composite score for the Copy clock. DCTclock Oscillatory Motion feature score for the Command clock, DCTclock Average Speed feature score for the Copy clock, and DCTclock Maximum Speed feature score for the Copy clock. A logistic regression classifier may be trained using the five features as inputs into a learning model, and produces a binary αβ± status as output. The actual αβ± status may be determined by using a cutoff threshold, such as a cutoff threshold of 1.185, on the raw value of Pittsburgh compound B (PiB) distribution volume ratio measurement for the neocortex brain region. The PiB is the PET detectable agent that binds to amyloid proteins in the brain and makes the proteins visible in the brain images. The specific combination of predictive features in the statistical model produces odds ratios for each predictor variable indicating the impact of each predictor variable on the binary outcome, αβ±. Resulting odds ratios can be incorporated into receiver operating characteristic (ROC) curves to determine the diagnostic capability of a binary classifier model. In experiments, this binary classifier model is able to predict αβ± status with an accuracy equal to or greater than existing tests with specificity based on the area under the curve (AUC) of 0.76 (sensitivity=0.65, specificity=0.73, total predictive accuracy=0.71).

TABLE 1
Exemplary Battery of Assessments

| Assessment | Definition |
|---|---|
| Delayed Recall accuracy score | The accuracy count of the 3-word recall test |
| | displayed in a score of 0-3. |
| DCTclock Spatial Reasoning | The spatial abilities demonstrated during |
| composite score | the process of drawing a copy of the |
| | displayed clock. This feature considers |
| | metrics pertaining to the geometric |
| | properties of the drawing including the |
| | circularity of the clock circle, placement of |
| | clock components, and drawing placement |
| | on the page. |
| DCTclock Oscillatory Motion | A measure of how much the motion of the |
| | pen deviates from a smooth pen motion |
| | during the drawing process of the copying |
| | of the clock. |
| Temporal Speech Features: | Speech timing features related to speech |
| Long and short pause count between | fluency and the general speed of the verbal |
| words | responses and reproductions of the words. |
| Time to first word | |
| Hesitations | |
| Pause time (%) | |
| Acoustic Voice Features: | Acoustic voice features associated with the |
| Frequency: average, range, StDev., jitter | loudness, pitch, and overall quality of the |
| Volume: average, range, StDev., | produced speech. |
| shimmer | |
| Spectral features: Cepstral peak | |
| prominence | |
| Voice time (%) | |
| Speech and Language Production Features: | Speech and production features related to |
| Filler words and sounds | language, words and sounds that are |
| % of filler words to relevant content | traditionally associated with the presence of |
| words | cognitive impairment. |
| Semantic language | |
| Relevant content units | |
| Eye Gaze and Sentiment Features: | Gaze tracking features produced from video |
| Spatial distribution of eye movements | recordings of the eye movements of the |
| Frequency and severity of fluctuations | patient undertaking the DCR (DCTclock |
| in eye movement | test + 3 word recall test). A deep learning |
| Fixation frequency and average duration | model analyzes the video recording frame- |
| Locating and response time in Copy | by-frame and produces a gaze and |
| Clock | sentiment prediction for each frame. Post- |
| Number of switches between prompt | processing algorithms analyze the gaze |
| and drawing areas in Copy Clock | predictions and produce the given gaze |
| Percentage time looking off screen | tracking features for the patient. |
| Average gaze distance ahead of stylus | |
| Stationary gaze entropy/transition gaze | |
| entropy | |
| Hemispheric visual neglect | |
| Blink rate | |
| Stylus Objective Feature: Pressure | The force of the touch, where a value of 1 |
| | represents the force of an average touch, a |
| | predetermined benchmark set by the stylus |
| | system. The force reported is measured |
| | along the axis of the pencil and is included |
| | into a deep learning model that produces a |
| | feature prediction for each patient. |
| Stylus Objective Feature: Azimuth | A measure of the direction in which the |
| | stylus is pointing in radians relative to the |
| | plane of the screen. The azimuth angle |
| | increases as the user swings the cap-end of |
| | the stylus in a clockwise direction around |
| | the tip included into a deep learning model |
| | that produces a feature prediction for each |
| | patient . |
| Stylus Objective Feature: Altitude | A measure of the elevation of the stylus |
| | above the plane of the screen in radians. A |
| | value of 0 radians indicates that the stylus is |
| | parallel to the surface, which increases as |
| | the user lifts the cap-end from the surface |
| | and is included into a deep learning model |
| | that produces a feature prediction for each |
| | patient. |
| Stylus Objective Feature: Stroke Count | The number of individual pen strokes |
| | produced in the drawing of either the |
| | command or copy clock, with each stroke |
| | delineated by each stylus point-end lift |
| | from the tablet surface and incorporated |
| | into a deep learning model that produces a |
| | feature prediction for each patient. |
| Linus Life and Health Questionnaire: | The Life and Health Questionnaire (LHQ) |
| Subjective/Patient-reported | is a brief digital survey that captures key |
| Depression/Anxiety | lifestyle and health factors for cognitive |
| Frailty | impairment/dementia. These questions have |
| Sleep | been demonstrated to significantly predict |
| Nutrition | functional status both in isolation and also |
| Physical Activity | when combined with other metrics listed |
| Social engagement | above. |
| Cognitive stimulation | |
| Life Satisfaction/Purpose | |

After a patient completes the Digital Clock and Recall (DCR) tests/assessment within the battery of assessments, the resulting raw data may be analyzed and metrics may be produced. Some of these metrics may provide quantitative performance on each segment (e.g., DCTclock score) while others of these metrics may measure various aspects of the patient's speech recordings on the Recall segment. It has been found that there are 12 metrics that correlate well with Aβ± status. These metrics include, but are not limited to:
Four speech jitter metrics (cycle-to-cycle variability of period duration of speech acoustic signal) from delayed recall (fully enumerated as deviation of fundamental frequency from one pitch period cycle to the other, i.e., local absolute, amount of periodic disturbance within five period cycles, “PPQ5”, relative average perturbation between period cycles, “RAP”, and difference of differences between period cycles, “DDP”);
Mean fundamental voice frequency (FO) from both immediate and delayed recall;
Decibel level variance and standard deviation for immediate recall;
Rate of speech (number of unique speech intervals divided by the total speech duration) on immediate recall;
DCTclock score (a number between 0 and 100 that represents a patient's overall cognitive performance as assessed by DCTclock);
DCTclock Spatial Reasoning composite score (a composite of metrics pertaining to the geometric properties of the clock drawing) for the copied clock; and
Age.

A machine learning model, such as a logistic regression classifier, may be trained using the 12 features as input. The machine learning model, such as a logistic regression classifier, may output a binary αβ± status. The actual αβ± status may be determined by using a cutoff threshold, such as a cutoff threshold of 0.089 (a published and tested threshold), on the concentration ratio of protein amyloids Aβ42 and Aβ40 (Aβ42/40) within a patient blood sample. In experiments, such a model may be capable of classifying αβ± status with an AUC (Area under the Receiver operating characteristic Curve) of 0.80 (sensitivity=0.75, specificity=0.7, total accuracy=0.71). The cognitive assessment battery may also include a Life and Health Questionnaire (LHQ). The LHQ may significantly predict mild and moderate deficits in functional activities of daily life (p's<0.001, R2=0.13, and R2=0.12) and may be considered in the prediction of ADRD related αβ± status.

The LHQ is based on evidence from prospective longitudinal trials that identify key lifestyle behaviors and psychosocial factors that either confer risk or protection from progression of cognitive impairment to dementia. The trials have demonstrated that factors such as physical activity, sleep, nutrition, cognitive engagement, purpose in life, social connectivity, frailty and vascular/metabolic risk factors significantly influence a patient's cognitive trajectory and risk of developing dementia in the future. Additionally, the LHQ reveals information about the individual patient's cognitive reserve, which refers to differences in cognitive processes or neural networks that enable some individuals to cope better with brain pathology. An exemplary set of questions included in the LHQ is shown in Table 2 below:

TABLE 2
Sample LHQ Questions

“I perform cognitively stimulating tasks (like reading, writing, drawing, or playing a musical
instrument) every day or almost every day.”
“I have lost more than 10 pounds over the last month without going on a diet.”
“I think that my energy level has gotten a lot lower over the last two or three years.”
“I drink an alcoholic beverage (like beer, wine, or spirits) every day or almost every day.”
“I smoke habitually.”
“I have felt more stressed than usual in the last month.”
“A medical doctor has diagnosed me with hypertension.”
“I am undergoing treatment for hypertension.”
“A medical doctor has diagnosed me with diabetes.”
“I am undergoing treatment for diabetes.”
“My doctor has told me that my current cholesterol levels are too high.”
“I have noticed (or other people have told me) that my hearing has gotten worse.”
“I have noticed that my sense of smell has gotten worse over the last two or three years.”
“On average, I eat more than two servings of fruit every day.”
“On average, I eat more than one serving of vegetables every day.”
“I eat red meat every day or almost every day.”
“On average, I sit more than six hours a day.”
“For one year or more, I have been doing some form of moderate (cycling, fast walking) or
vigorous exercise (running, spinning, playing basketball) at least three times a week.”
“It usually takes me more than thirty minutes to fall asleep.”
“I often wake up during the night and stay awake for more than 30 minutes.”
“I sleep on average between 7 and 8 hours each night.”
“I sometimes feel lonely or that I lack company.”
“I am involved in some kind of association, club, choir or volunteer activity at least once a
week.”
“I know what I want to achieve in life, and what is my purpose.”
“I feel satisfied with my life.”
“I am generally satisfied with the course my life has taken.”

In some embodiments, a biomarker is predicted using a machine learning model. The algorithm in the prediction machine learning model may assign a different weight to each feature in an assessment within the battery of assessment's input according to its predictive value on the amyloid positivity target variable. Weights are not assigned manually but are learned by the model as part of the model training process. Self-reported answers from the LHQ each factor in as a predictor variable, much like acoustic and temporal speech, individual pen or stylus metrics, and recall metrics.

In some embodiments, the actual αβ± status may be determined by using a cutoff threshold, such as a cutoff threshold of 1.185, or greater or less than, as needed, on the raw value of Pittsburgh compound B (PiB) distribution volume ratio measurement for the neocortex brain region. The PiB is the PET detectable agent that binds to amyloid proteins in the brain and makes the proteins visible in the brain images. The specific combination of predictive features in the statistical model produces odds ratios for each predictor variable indicating the impact of each predictor variable on the binary outcome αβ±. Resulting odds ratios can be incorporated into receiver operating characteristic (ROC) curves to determine the diagnostic capability of a binary classifier model. In experiments, such a model is able to predict αβ± status with an accuracy equal to or greater than existing tests with specificity based on the area under the curve (AUC) of 0.76 (sensitivity=0.65, specificity=0.73, total predictive accuracy=0.71).

In some embodiments, the actual αβ± status may be determined by using a cutoff threshold, such as a cutoff threshold of 0.089 (a published and tested threshold), on the concentration ratio of protein amyloids Aβ42 and Aβ40 (Aβ42/40) within a patient blood sample. In experiments, this model is capable of classifying αβ± status with an AUC (Area under the Receiver operating characteristic Curve) of 0.80 (sensitivity=0.75, specificity=0.7, total accuracy=0.71). The cognitive assessment battery may also include the LHQ. The LHQ significantly predicts mild and moderate deficits in functional activities of daily life (p's<0.001, R2=0.13, and R2=0.12) and is considered in the prediction of ADRD related αβ± status.

In some embodiments the prediction (feature/prediction) is provided to the recommendation engine to determine a recommendation unique to the patient. The intervention output can take the form of a table listing various scores including cognition, motor skills, mood, and voice. As shown in the table below, the clinician can be provided with an output table, as shown in Table 3, with a basis for the recommendation provided.

TABLE 3
Example Recommendation

| Category: | Cognition | Motor | Mood | Voice |
|---|---|---|---|---|
| Metrics | | Mostly Timing | | |
| Scoring | 0 points = ≥1 SD | 0 points = ≥1 SD | 0 points = <1 SD | ±0 points = <1 SD |
| | 1 Points = <1 SD | 1 Points = <1 SD | 1 Points = >1 SD | ±1 Points = ≥1 SD |
| Recommendation(s) | General | Motor | LHQ-based Mood | ±NEW |
| affected | Impairment | Recommend- | Recommendation | Recommendation: |
| | Recommend- | ation (General - | | This would be its |
| | ations | Parkinsonism, | | own |
| | (Red/Yellow/ | Essential | | recommendation, |
| | Green) | Tremor) | | and could be |
| | | | | something related |
| | | | | to respiration, |
| | | | | motor, or just voice |
| | | | | quality |
| | | | | ±“We recommend |
| | | | | an evaluation of the |
| | | | | airway, respiration, |
| | | | | and potential |
| | | | | laryngeal imaging |
| | | | | depending on the |
| | | | | patient's medical |
| | | | | history and |
| | | | | concurrent |
| | | | | recommendations.” |
| | | | | More than 1 point |
| | | | | in this domain |
| | | | | would trigger the |
| | | | | recommendation |
| | | | | for a voice |
| | | | | evaluation |
| Recommendation | More than 4 | More than 6 | More than 4 | |
| Rule | points in this | points in this | points in this | |
| | domain would | domain would | domain would | |
| | create a strong | create a strong | create a strong | |
| | cognitive | motor | mood | |
| | recommendation | recommendation | recommendation | |
| | in conjunction | in conjunction | in conjunction | |
| | with a yellow or | with a motor | with a mood | |
| | red recommend- | recommendation | recommendation | |
| | ation from the | from the DCR. | from the LHQ/or | |
| | DCR. | | mood assessment. | |

Each recommendation may be output with a score across four categories: cognition, motor, mood, and voice. Related metrics are described below each category, as is the scoring system used with the category. The recommendation affected by the category is shown in the above Table 3 along with any subsequent recommendation to consider. For example, a general impairment recommendation within the cognitive category can trigger a recommendation in the mood category. The rules for the recommendation are also included, along with any relevant information for related recommendations.

FIG. 2 illustrates an example process for data used to train a machine learning model. In workflow 200, multimodal data may be collected by a system including a touchscreen, webcam, stylus, and microphone at step 202. Data can be collected by one or more components of the system, as is needed. The collected data may represent patient responses to the battery of assessments conducted by the system. At step 204, the modality-specific data may be processed, where data can be organized by the modality in which it was collected. For example, and as shown in the step 204 graphic, responses from the webcam may be grouped with other webcam responses, and separated from touchscreen responses.

At the patient data model step 206, first and second order features of the processed data are extracted. First order features include, for example, stroke based drawing features and time based drawing features from the touchscreen data, as well as eye tracking features and sentiment/emotion features from the webcam feature data. Additionally, stylus orientation features as well as stylus force/grip strength features data from the stylus and speech content and speech aural quality features from the microphone may be considered first order features. Second order features can, for example, include data embedded within each modality by which the patient provides a response. The patient data model can also include aggregated clinician codes associated with a patient.

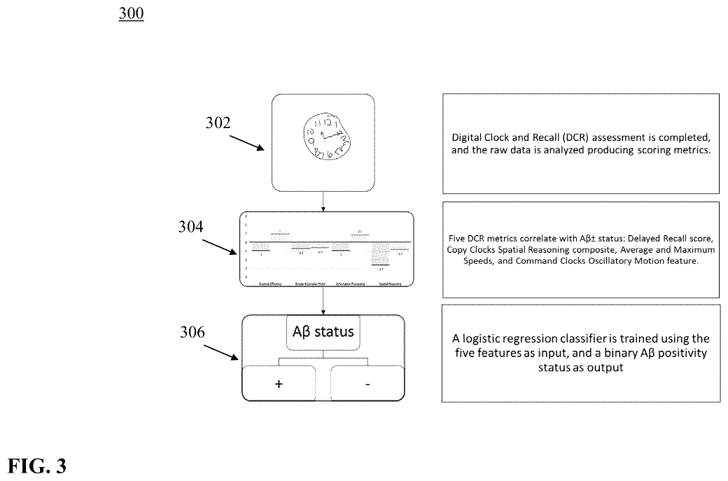

FIG. 3 is a further example workflow 300 of predicting a binary αβ± status. Once a patient finishes a DCR assessment at step 302, the raw data may be analyzed to produce scoring metrics as described herein. At step 304, five metrics shown to correlate with Aβ status, which include Delayed Recall status. Copy Clocks Spatial Reasoning composite. Average and Maximum Speeds, and Command Clocks Oscillatory Motion feature. The resulting scores from these tests/assessments may then be provided as inputs to a machine learning model, such as a logistic regression classifier, at step 306. The output from the machine learning model, such as the classifier, may be a binary Aβ± status.

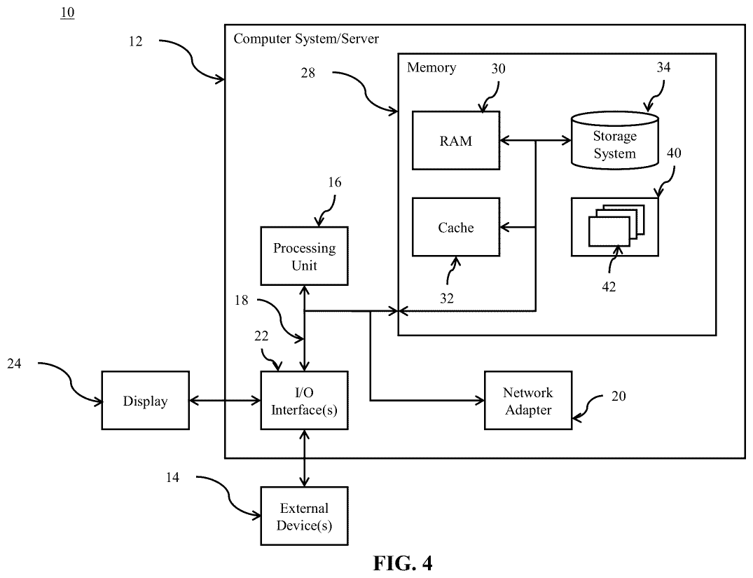

Referring now to FIG. 4, a schematic of an example of a computing node is shown. Computing node 10 is only one example of a suitable computing node and is not intended to suggest any limitation as to the scope of use or functionality of embodiments described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems. set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 4, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM. DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the disclosure.

Program/utility 40. having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments as described herein.

Computer system/server 12 may also communicate with one or more external devices 14 such as a keyboard, a pointing device, a display 24. etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture. functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed:

1. A method for predicting a biomarker status, comprising:
administering a battery of assessments to a patient;
collecting multimodal data based on one or more responses to the battery of assessments from the patient;
extracting one or more feature sets from the one or more response;
providing the one or more feature sets to a trained machine learning model;
predicting, using the trained machine learning model, a status of a biomarker of the patient;
providing the prediction into a recommendation engine;
determining one or more interventions based on the prediction, wherein the one or more interventions include values to the patient;
providing the one or more interventions as output.

2. The method of claim 1, wherein the one or more feature is extracted using a first order measure.

3. The method of claim 2, wherein a first order measure comprises a feature extracted from the response and organized by a modality associated with the feature.

4. The method of claim 1, wherein the one or more feature is extracted using a second order measure.

5. The method of claim 4, wherein a second order measure comprises extracting an embedded characteristics of the response and associating the characteristics with a modality.

6. The method of claim 1, further comprising determining a health condition based on the status of the biomarker.

7. The method of claim 1, wherein determining the one or more interventions comprises thresholding the biomarker status.

8. The method of claim 1, wherein the one or more interventions include a holistic state of the prediction, the holistic state of the prediction comprising:
receiving additional data associated with the patient; and
weighing the prediction with the additional data.

9. The method of claim 1, wherein the trained machine learning model is trained with a subset of feature sets as an input.

10. The method of claim 9, wherein the subset of feature sets includes a delayed recall score, a composite clock score, an average speed score, an oscillatory motion feature score, and a maximum speed score.

11. The method of claim 1, wherein the trained machine learning model is a regression model.

12. The method of claim 11, wherein the regression model is a logistic regression classifier.

13. The method of claim 1, wherein the battery of assessments includes at least one of a digital clock and recall assessment and a DCTclock assessment.

14. The method of claim 1, wherein the collecting the multimodal data comprises collecting data from at least a touchscreen, a microphone, a webcam, and/or a stylus.

15. The method of claim 1, wherein the biomarker comprises a beta-amyloid.

16. The method of claim 1, wherein providing the one or more interventions as output comprises transmitting the one or more interventions to a computing device associated with a clinician.

17. The method of claim 1, wherein the one or more interventions comprise a score for a plurality of categories and a recommendation for each category.

18. The method of claim 1, wherein the recommendation includes a physical or mental evaluation of the patient.

19. The method of claim 1, further comprising receiving patient demographic and medical history from the patient.

20. The method of claim 1, wherein the battery of assessments is conducted on a mobile computing device.

21. A system for predicting amyloid biomarker status, the system comprising:
a computing node comprising a computer readable storage medium having program instructions embodied therewith, the program instructions executable by a processor of the computing node to cause the processor to perform a method comprising:
administering a battery of assessments to a patient;
collecting multimodal data based on one or more responses to the battery of assessments from the patient;
extracting one or more feature sets from the one or more response;
providing the one or more feature sets to a trained machine learning model;
predicting, using the trained machine learning model, a status of a biomarker of the patient;
providing the prediction into a recommendation engine;
determining one or more interventions based on the prediction, wherein the one or more interventions include values to the patient;
providing the one or more interventions as output.

22. A computer program product for predicting amyloid biomarker status, the computer program product comprising a computer readable storage medium having program instructions embodied therewith, the program instructions executable by a processor to cause the processor to perform a method comprising:
administering a battery of assessments to a patient;
collecting multimodal data based on one or more responses to the battery of assessments from the patient;
extracting one or more feature sets from the one or more response;
providing the one or more feature sets to a trained machine learning model;
predicting. using the trained machine learning model, a status of a biomarker of the patient;
providing the prediction into a recommendation engine;
determining one or more interventions based on the prediction, wherein the one or more interventions include values to the patient;
providing the one or more interventions as output.
